(19) United States
(12) Patent Application Publication
(10) Pub. No.: US 2020/0237736 A1
(43) Pub. Date: Jul. 30, 2020

(54) METHODS FOR PREVENTING OR TREATING CANCER RESISTANCE TO EGFR INHIBITION

(71) Applicants: INSERM (INSTITUT NATIONAL DE LA SANTE ET DE LA RECHERCHE MEDICALE), Paris (FR); UNIVERSITE DE ROUEN NORMANDIE, Mont-Saint-Aignan (FR); ICAHN SCHOOL OF MEDICINE OF MOUNT SINAI, New York, NY (US)

(72) Inventors: Luca GRUMOLATO, Mont-Saint-Aignan (FR); Alexis GUERNET, Mont-Saint-Aignan (FR); Stuart AARONSON, New York, NY (US); Youssef ANOUAR, Mont-Saint-Aignan (FR)

(21) Appl. No.: 16/624,985

(22) PCT Filed: Jun. 22, 2018
(86) PCT No.: PCT/EP2018/066803

§ 371 Date: Dec. 20, 2019

(30) Foreign Application Priority Data
Jun. 23, 2017 (EP) 17305784.5

(51) Int. Cl. A61K 31/44; A61K 31/5377; A61K 31/519; A61K 31/506; A61K 39/395; A61P 35/00

(52) U.S. Cl. CPC A61K 31/44; A61K 31/5377; A61P 35/00; A61K 31/506; A61K 39/3955; A61K 31/519

(57) ABSTRACT

The invention relates to methods for preventing or treating resistance to EGFR inhibitors in cancer patients. More particularly, after performing a screen for small molecules potentially capable of countering cancer resistance to EGFR TKI, inventors identified the multikinase inhibitor sorafenib, which has the property, in combination with EGFR TKI, to prevent the enrichment of tumor cells containing mutations responsible for NSCLC resistance to first and third generation EGFR TKI. These data indicate that this multikinase inhibitor can prevent resistance to several generations of EGFR TKI. These in vitro data were confirmed in an in vivo xenograft mouse model of NSCLC. Finally these data were reproduced in cancer cells using SC-1, a sorafenib analogue. Accordingly, the invention relates to a method of preventing and/or treating cancer resistance to treatment with an epidermal growth factor receptor (EGFR) inhibitor in a subject in need thereof comprising administering to the subject sorafenib drug or sorafenib analogue, alone or in combination with an EGFR inhibitor.

### FIELD OF THE INVENTION

The invention relates to methods for preventing or treating cancer resistance to EGFR inhibitors.

### BACKGROUND OF THE INVENTION

The epidermal growth factor receptor (EGFR) pathway is crucial in the development and progression of human epithelial cancers. The treatment with EGFR inhibitors has a synergistic growth inhibitory and pro-apoptotic activity in different human cancer cells which possess a functional EGFR-dependent autocrine growth pathway through to a more efficient and sustained inhibition of Akt and/or MAPK.

EGFR inhibitors have been approved or tested for treatment of a variety of cancers, including non-small cell lung cancer (NSCLC), head and neck cancer, colorectal carcinoma, and Her2-positive breast cancer, and are increasingly being added to standard therapy. EGFR inhibitors, which may target either the intracellular tyrosine kinase domain or the extracellular domain of the EGFR target, are generally plagued by low population response rates, leading to ineffective or non-optimal chemotherapy in many instances, as well as unnecessary drug toxicity and expense. For example, a reported clinical response rate for treatment of colorectal carcinoma with cetuximab (a chimeric monoclonal antibody targeting the extracellular domain of EGFR) is about 11% (Cunningham et al, N Engl J Med 2004; 351: 337-45), and a reported clinical response rate for treatment of NSCLC with erlotinib is about 8.9% (Shepherd F A, et al, N Engl J Med 2005; 353:123-132). Since the identification of activating mutations of the epidermal growth factor receptor (EGFR) in NSCLC (Lynch et al., N Engl J Med 2004; 2004; 350: 2129-2139), patients whose tumor displays such mutations are treated with EGFR TKI (tyrosine kinase inhibitors), resulting in a dramatic increase in the clinical response rate (Chong and Janne, Nat Med 2013; 19: 1389-1400). However, a resistance mechanism almost invariably occurs when EGFR inhibitors are used in the treatment of this type of patients.

In France, lung cancer is the leading cause of cancer death (Institut National du Cancer). Non-small-cell lung cancers (NSCLCs) constitute about 85% of lung malignancies and include adenocarcinomas, squamous cell carcinomas and large cell carcinomas. Different genetic aberrations that can drive NSCLC have been identified. These oncogenic mutations, which are generally not redundant, constitute potential targets for therapy. Indeed, patients whose tumors contain activating mutations of the epidermal growth factor receptor (EGFR), which are present in roughly 12% of NSCLCs, are treated with specific TKIs, including gefitinib (Iressa, AstraZeneca), erlotinib (Tarceva, Genentech) and afatinib (Gilotrif, Boehringer Ingelheim) (Tsao et al., 2016). Unfortunately, while these tumors generally show a remarkable response to EGFR TKI, they invariably relapse, as a result of the amplification of small subpopulations of resistant clones, which are generally already present before the onset of the treatment (Bhang et al., 2015; Hata et al., 2016). The main mechanisms of resistance to these compounds identified so far include a secondary mutation in EGFR kinase domain (EGFR-T790M), found in 50-60% of patients, amplification of other receptor tyrosine kinases, such as MET and HER2, and histologic changes, including epithelial to mesenchymal transition or transformation into small cell carcinoma (Chong and Janne, 2013; Cortot and Janne, 2014). Recent clinical trials have shown efficacy of third generation irreversible EGFR TKI against NSCLCs that developed resistance to gefitinib/erlotinib through the T790M mutation (Janne et al., 2015; Sequist et al., 2015). These studies resulted in the 2015 approval of one of these compounds, osimertinib (Tagrisso, AstraZeneca) for the treatment of patients with metastatic EGFR-T790M mutation-positive NSCLC, who have progressed on or after EGFR TKI therapy. However, after an initial response, the tumors become resistant to this new drug and relapse. The most common mechanism identified so far involves substitution of a cysteine with a serine in position 797 of the receptor, which corresponds to the residue that is covalently bound by osimertinib (Minari et al., 2016; Thress et al., 2015).

Thus, there is a need for a new strategy for preventing or treating cancer resistance to EGFR inhibitors, so as to better individualize patient therapy.

The invention addresses these needs, as it relates to methods and treatment approaches useful in the prevention and treatment of EGFR inhibitor-resistant cancer.

### SUMMARY OF THE INVENTION

In a first aspect, the invention relates to a method of treating a cancer with an acquired resistance to treatment with a epidermal growth factor receptor (EGFR) inhibitor in a subject in need thereof comprising administering to the subject sorafenib drug or sorafenib analogue.

In a second aspect, the invention relates to a method of preventing and/or treating cancer acquired resistance to treatment with an epidermal growth factor receptor (EGFR) inhibitor in a subject in need thereof comprising administering to the subject a combination of drugs selected from the group consisting of a sorafenib drug or sorafenib analogue and an EGFR inhibitor.

In a third aspect, the invention relates to a method of preventing emergence of resistance to treatment with an epidermal growth factor receptor (EGFR) inhibitor in a subject in need thereof comprising administering to the subject a combination of sorafenib drug or a sorafenib analogue together with a EGFR inhibitor.

### DETAILED DESCRIPTION OF THE INVENTION

Taking advantage of the CRISPR-barcoding approach previously developed by the inventors (Guernet et al., 2016), inventors performed a screen for small molecules potentially capable of inhibiting cancer resistance to EGFR TKI such as in a non-small cell lung cancer (NSCLC). Among the different compounds tested, the multikinase inhibitor sorafenib dose-dependently prevented the enrichment of PC9 cells containing the EGFR-T790M mutation in the presence of gefitinib (FIG. 1A). Consistent with these data, co-treatment with sorafenib abolished the formation of resistant colonies in the presence of gefitinib (FIG. 1B). Similar results were obtained in other EGFR TKI-sensitive NSCLC cells, including HCC827 and HCC4006 (FIG. 2). To investigate the effects of sorafenib on NSCLC resistance to third generation EGFR TKI, inventors generated through CRISPR-barcoding a small subset of clones containing the EGFR-C797S mutation within a mass population of PC9-(EGFR-T790M) cells previously selected in the presence of gefitinib. Treatment with osimertinib induced a strong amplification of EGFR-C797S cells, which was blocked by co-treatment with sorafenib. These data indicate that this multikinase inhibitor can also prevent NSCLC resistance to latest generation EGFR TKIs.

To confirm the in vitro data, EGFR-T790M PC9 cells containing the EGFR-C797S CRISPR-barcode were subcutaneously and bilaterally injected in immunocompromised mice. Four weeks after inoculation, when the volume of the tumors reached 40-50 mm3, the mice were treated in the presence or the absence of osimertinib (5 mg/kg); sorafenib (60 mg/kg) or a combination of the two drugs. In the presence of sorafenib or osimertinib, the growth of the tumors was initially inhibited, but it eventually resumed after a few weeks. Conversely, combination of these two drugs provoked a decrease of tumor size, which remained stable for two months, when the remaining mice were sacrificed. These results are consistent with the previous data and indicate that sorafenib co-treatment can prevent NSCLC cell resistance to EGFR TKI in vivo. These data also indicate that osimertinib treatment eliminated most, if not all, sensitive cells, while the growth of EGFR-C797S resistant cells was arrested by sorafenib.

Finally these data were reproduced in PC9 cells using the multikinase inhibitors SC-1 a sorafenib analogue (Example 2 FIG. 12).

Based on the present results, the inventors propose a new therapeutic approach to prevent the emergence of cancer resistance to EGFR inhibitors or to treat a cancer that has developed resistance to an EGFR inhibitor.

Accordingly, a first aspect of the invention relates to a method of treating a cancer with an acquired resistance to treatment with an epidermal growth factor receptor (EGFR) inhibitor in a subject in need thereof comprising administering to the subject a sorafenib drug or a sorafenib analogue.

A second aspect of the invention relates to a method of preventing and/or treating cancer acquired resistance to treatment with an epidermal growth factor receptor (EGFR) inhibitor in a subject in need thereof comprising administering to the subject combination of drugs selected from the group consisting of a sorafenib drug or a sorafenib analogue and an EGFR inhibitor.

A third aspect of the invention relates to a a method of preventing emergence of resistance to treatment with an epidermal growth factor receptor (EGFR) inhibitor in a subject in need thereof comprising administering to the subject a combination of sorafenib drug or a sorafenib analogue together with a EGFR inhibitor

As used herein, the term “sorafenib” (also marked as Nexavar®) refers to a member of a family of aryl ureas compounds, initially reported as being a RAF kinase inhibitors (see WO0042012 and U.S. Pat. Nos. 7,235,576, 8,124,630, 8,618,141, 8,841,330) approved by FDA for the treatment of primary kidney cancer (advanced renal cell carcinoma) in 2005, advanced primary liver cancer (hepatocellular carcinoma) in 2007, and radioactive iodine resistant advanced thyroid carcinoma in 2013. Sorafenib inhibits several tyrosine kinases, including VEGFR, PDGFR, c-Kit and Ret, as well as the serine/threonine kinase RAF (Wilhelm et al., 2006). Accordingly Sorafenib inhibits cellular signaling via a variety of receptors which play a role in tumor angiogenesis and tumor cell proliferation. Hence, the simultaneous inhibition of these receptors promotes reduced tumor vascularization and cancer cell death. Sorafenib has the following structure:

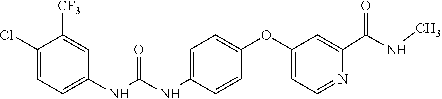 [SMILES: CNC(=O)c1cc(Oc2ccc(NC(=O)Nc3ccc(Cl)c(C(F)(F)F)c3)cc2)ccn1]

As used herein, the term “analogue of sorafenib” refers to family members of aryl ureas compounds described in WO0042012 patent which have a RAF kinase inhibitors activity and similar structure as sorafenib all of which are herein incorporated by reference.

Examples of such analogues are the following:
N-(2-methoxy-5-(trifiuoromethyl) phenyl)-N′-(4(2-(Nmethylcarbamoyl)-4-pyridyloxy) phenyl) urea of the formula:

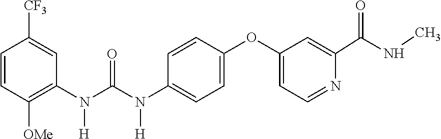 [SMILES: CNC(=O)c1cc(Oc2ccc(NC(=O)Nc3cc(C(F)(F)F)ccc3OC)cc2)ccn1]

N-(4-chloro-3-(trifiuoromethyl) phenyl)-N′-(4(2-(Ncarbamoyl)-4-pyridyloxy) phenyl) urea of the formula:

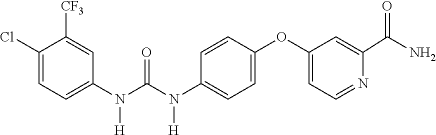 [SMILES: NC(=O)c1cc(Oc2ccc(NC(=O)Nc3ccc(Cl)c(C(F)(F)F)c3)cc2)ccn1]

N-(2-methoxy-4-chloro-5-(trifloromethyl) phenyl)-N′-(3(2-(N-methylcarbamoyl)-4-pyridyloxy) phenyl) urea of the formula:

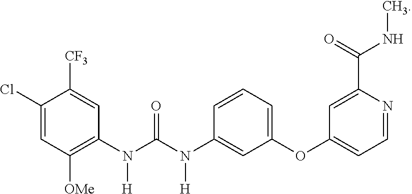 [SMILES: CNC(=O)c1cc(Oc2cccc(NC(=O)Nc3cc(C(F)(F)F)c(Cl)cc3OC)c2)ccn1]

The term “analogue of sorafenib” also refers the compound SC-1 (also called N-[4-Chloro-3-(trifluoromethyl)phenyl]-N′-[4-(4-cyanophenoxy)phenyl]-urea ref. CSID:26608398: www.chemspider.com/Chemical-Structure.26608398.html). SC-1 is a derivative of the multiple tyrosine kinase inhibitor sorafenib with no kinase-inhibition activity. SC-1 blocks STATS phosphorylation and activation with similar potency to sorafenib, and induces apoptosis in breast cancer cell lines (Liu et al. Breast Cancer Research 2013, 15:R63) and EGFR wild-type NSCLC cell lines (Wang et al J of Thor Onc.2014, 9: 488-496). SC-1 has the following structure:

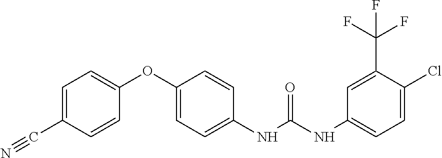 [SMILES: N#Cc1ccc(Oc2ccc(NC(=O)Nc3ccc(Cl)c(C(F)(F)F)c3)cc2)cc1]

The “subject” or “patient” may be any mammal, preferably a human being, whatever its age or sex. The patient is afflicted with a cancer. The subject or patient may be already subjected to a treatment, by any EGFR inhibitor.

The cancer is a cancer in which the signaling pathway through EGFR is involved. In particular, it may be e.g. colorectal, lung, breast, ovarian, endometrial, thyroid, nasopharynx, prostate, head and neck, kidney, pancreas, bladder, glioma or brain cancer (Ciardello F et al. N Engl J Med. 2008 Mar. 13; 358(11):1160-74; Wheeler D L et al. Nat Rev Clin Oncol. 2010 September; 7(9): 493-507; Zeineldin R et al. J Oncol. 2010; 2010:414676; Albitar L et al. Mol Cancer 2010; 9:166; Leslie K K et al. Gynecol Oncol. 2012 November; 127(2):345-50; Mimeault M et al. PLoS One. 2012; 7(2):e31919; Liebner D A et al. Ther Adv Endocrinol Metab. 2011 October; 2(5):173-95; Leboulleux S et al. Lancet Oncol. 2012 September; 13(9):897-905; Pan J et al. Head Neck. 2012 Sep. 13; Chan S L et al. Expert OpinTher Targets. 2012 March; 16 Suppl 1:S63-8; Chu H et al. Mutagenesis. 2012 Oct. 15; Li Y et al. Oncol Rep. 2010 October; 24(4):1019-28; Thomasson M et al. Br J Cancer 2003, 89:1285-1289; Thomasson M et al. BMC Res Notes. 2012 May 3; 5:216). In certain embodiments, the tumor is a solid tissue tumor and/or is epithelial in nature. For example, the patient may be a colorectal carcinoma patient, a non-small cell lung cancer (NSCLC) patient, a head and neck cancer patient (in particular a squamous-cell carcinoma of the head and neck patient), a glioma patient, a pancreatic cancer patient, or an endometrial cancer patient. More particularly, the patient may be a colorectal carcinoma patient, a lung cancer (in particular a NSCLC) patient, a head and neck cancer patient (in particular a squamous-cell carcinoma of the head and neck patient), or a pancreatic cancer patient.

In a preferred embodiment, the cancer is a lung cancer, still preferably the cancer is a non-small cell lung cancer (NSCLC). Indeed, data presented in Examples, clearly indicate that sorafenib may be used to treat or prevent cancer resistance to several generation of EGFR inhibitors (and in particular EGFR tyrosine kinase inhibitor (TKI) such as gefitinib and osimertinib) in NSCLC.

Preferably in lung cancer, especially in NSCLC the patient has a first activating mutation in EGFR kinase domain, selected from the group consisting of exon 19 deletions, L858R, exon 20 insertions, G719X, L861X, exon 19 insertions (Chong and Janne, Nat Med 2013; 19: 1389-1400). More preferably the patient has a first activating mutation in EGFR kinase domain and a secondary or a secondary and a tertiary mutation in EGFR kinase domain selected from the group consisting of T790M or C797S.

These results, obtained in a cancer (NSCLC) in which the EGFR signaling pathway is known to be involved, clearly suggest that sorafenib might be used to treat or prevent cancer resistance to EGFR inhibitors (and in particular EGFR tyrosine kinase inhibitor (TKI) such as gefitinib and osimertinib) in any other cancer in which the EGFR signaling pathway is known to be involved, such as colorectal, ovarian, endometrial, thyroid, nasopharynx, prostate, head and neck, kidney, pancreas, bladder, glioma or brain cancer. Since sorafenib is able to treat or prevent resistance to EGFR inhibitors in one type cancer in which EGFR signaling pathway is known to be involved, use of sorafenib can be reasonably expected to be useful in any other cancer in which the EGFR signaling pathway is known to be involved.

In still another preferred embodiment, the cancer is a colorectal cancer, in particular a metastatic colorectal cancer.

Preferably in colorectal cancer, the patient has a KRAS wild type tumor, i.e., the KRAS gene in the tumor of the patient is not mutated in codon 12, 13 (exon 1), or 61 (exon 3). In other words, the KRAS gene is wild-type on codons 12, 13 and 61.

Wild type, i.e. non mutated, codons 12, 13 (exon 1), and 61 (exon 3) respectively correspond to glycine (Gly, codon 12), glycine (Gly, codon 13), and glutamine (Gln, codon 61). The wild-type reference KRAS amino acid sequence may be found in Genbank accession number NP_004976.2.

Especially the KRAS gene of the patient's tumor does not show any of the following mutations (Bos. Cancer Res 1989; 49:4682-4689; Edkins et al. Cancer Biol Ther. 2006; 5(8): 928-932; Demiralay et al. Surgical Science, 2012, 3, 111-115):

Any method known in the art may be used to assess the KRAS status of the patient. Preferably in colorectal cancer patients, the tumor can display mutations in EGFR extracellular domain, including S464L, G465R/E, K467T, I491M, S492R or R451C, known to confer resistance to anti-EGFR monoclonal antibodies (Arena et al., Clin Cancer Res 2015; 21: 2157-2166; Arena et al., Sci Transl Med 2016; 8(324):324ra14).

In still another preferred embodiment, the cancer is a pancreatic cancer.

As used herein, the term “treatment” refers to an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. Within the context of the invention, the term “treatment” may also mean prolonging survival as compared to expected survival if not receiving treatment.

The term “epidermal growth factor receptor”, “EGFR”, “ErbB” or “HER” refers to a receptor protein tyrosine kinase which belongs, to the ErbB receptor family and includes ErbB1 (or HER1 or EGFR), ErbB2 (or HER2), ErbB3 (or HER 3) and ErbB4 (or HER 4) receptors (Ullrich, 1984). The ErbB receptor will generally comprise an extracellular domain, which may bind an EGFR ligand; a lipophilic transmembrane domain; a conserved intracellular tyrosine kinase domain; and a carboxyl-terminal signaling domain harboring several tyrosine residues which may be phosphorylated. Being activated by their six structurally related agonists-EGF, tumor growth factor α (TGFα), heparin-binding EGF-like growth factor (HB-EGF), amphiregulin, betacellulin and epiregulin- the receptors promote pathways entailing proliferation and transformation. Activated EGFRs homo- or heterodimerize and subsequently autophosphorylation of cytoplasmic tyrosine residues is initiated. These phosphorylated amino acids represent docking sites for a variety of different proteins (Prenzel 2001). Tyrosine phosphorylation of the EGFR leads to the recruitment of diverse signaling proteins, including the Adaptor proteins GRB2 (Growth Factor Receptor-Bound Protein-2) and Nck (Nck Adaptor Protein), PLC-Gamma (Phospholipase-C-Gamma), SHC (Src Homology-2 Domain Containing Transforming Protein), and STATS (Signal Transducer and Activator of Transcription 5).

The expressions “epidermal growth factor receptor” “ErbB 1” and “HER1” and “EGFR” are used interchangeably herein and refer to human EGFR protein.

The term “EGFR inhibitor” or “ErbB inhibitor” refers to any EGFR inhibitor that is currently known in the art or that will be identified in the future, and includes any chemical entity that, upon administration to a patient, results in inhibition of a biological activity associated with activation of the EGFR in the patient, including any of the downstream biological effects otherwise resulting from the binding to EGFR of its natural ligand. Such EGFR antagonist include any agent (chemical entity, anti-EGFR antibody, . . . ) that may block EGFR activation or any of the downstream biological effects of EGFR activation. Such an inhibitor may act by binding directly to the intracellular domain of the receptor and inhibiting its kinase activity. Alternatively, such an antagonist may act by occupying the ligand binding site or a portion thereof of the EGFR, thereby making the receptor inaccessible to its natural ligand so that its normal biological activity is prevented or reduced. Alternatively, such an inhibitor acts by modulating the dimerization of ErbB polypeptides, or interaction of ErbB polypeptide with other proteins. Therefore the term “EGFR inhibitor” or “Erb1 inhibitor” or “HER1 inhibitor” refers to an antagonist of the EGFR protein.

Examples of EGFR inhibitor include but are not limited to any of the EGFR antagonists described in Garafalo S. et al. (Exp Opin. Ther Pat 2008) all of which are herein incorporated by reference.

The EGRF inhibitor may be an EGFR tyrosine kinase inhibitor (TKI), or may alternatively target the extracellular domain of the EGFR target.

In a preferred embodiment the cancer with an acquired resistance to treatment with an EGFR inhibitor is a cancer with an acquired resistance to treatment with an epidermal growth factor receptor (EGFR) tyrosine kinase inhibitor (TKI).

As used herein, the term “epidermal growth factor receptor (EGFR) tyrosine kinase inhibitor (TKI)” refers to a compound which leads to the intracellular inhibition of EGFR signaling pathway by targeting the intracellular kinase domains of the EGFRs.

Preferably the EGFR inhibitor is an EGFR tyrosine kinase inhibitor

In certain embodiments, the EGFR inhibitor is an EGFR tyrosine kinase inhibitor such as Erlotinib, Gefitinib, Lapatinib, Afatinib or Osimertinib.

EGFR tyrosine kinase inhibitors are mainly used in the treatment of lung cancer (in particular non small cell lung cancer, NSCLC), so that if the patient's cancer is lung cancer (in particular non small cell lung cancer, NSCLC), then the method according to the invention may preferably be used to treat or prevent cancer resistance to EGFR tyrosine kinase inhibitors, such as Erlotinib, Gefitinib, Lapatinib, Afatinib or Osimertinib.

Erlotinib, Gefitinib, Afatinib, Lapatinib and Osimertinib (mereletinib or AZD9291; trade name Tagrisso) are currently the clinically mostly used tyrosine kinase EGFR inhibitors. However, further EGFR tyrosine kinase inhibitors are in development, such as rociletinib, brigatinib (Alunbrig), Canertinib (CI-1033), Neratinib (HKI-272), Dacomitinib (PF299804, PF-00299804), TAK-285, AST-1306, ARRY334543, AG-1478 (Tyrphostin AG-1478), AV-412, OSI-420 (DesmethylErlotinib), AZD8931, AEE788 (NVP-5 AEE788), Pelitinib (EKB-569), CUDC-101, AG 490, PD153035 HCl, XL647, and BMS-599626 (AC480). The method according to the invention may also be used to treat cancer resistant to these tyrosine kinase EGFR inhibitors or any other tyrosine kinase EGFR inhibitors that might be further developed, in particular if the patient is suffering from lung cancer (in particular non small cell lung cancer, NSCLC), pancreatic cancer, glioma or brain cancer, or head and neck cancer (in particular squamous cell carcinoma of the head and neck (SCCHN)).

In another embodiment, the EGFR inhibitor is a molecule that targets the EGFR extracellular domain.

Molecules that target the EGFR extracellular domain, including anti-EGFR monoclonal antibodies such as Cetuximab or Panitumumab, are mainly used in the treatment of colorectal cancer or squamous cell carcinoma of the head and neck. As a result, if the patient's cancer is colorectal cancer (in particular metastatic colorectal cancer) or squamous cell carcinoma of the head and neck, then the method according to the invention may be used to predict response to molecules that target the EGFR extracellular domain, and in particular to anti-EGFR monoclonal antibodies, such as Cetuximab or Panitumumab.

Cetuximab and Panitumumab are currently the clinically mostly used anti-EGFR monoclonal antibodies. However, further anti-EGFR monoclonal antibodies are in development, such as Nimotuzumab (TheraCIM-h-R3), Matuzumab (EMD 72000), MM-151 and Zalutumumab (HuMax-EGFr). The method according to the invention may also be used to treat or prevent cancer resistance to these anti-EGFR monoclonal antibodies or any other anti-EGFR monoclonal antibodies (including fragments) that might be further developed, in particular if the patient is suffering from colorectal cancer (in particular metastatic colorectal cancer), glioma or brain cancer, pancreatic cancer or head and neck cancer (in particular squamous cell carcinoma of the head and neck (SCCHN))

In pancreatic cancer or head and neck cancer (in particular squamous cell carcinoma of the head and neck (SCCHN)), both tyrosine kinase EGFR inhibitors and anti-EGFR monoclonal antibodies are being tested as therapy, so that if the patient's cancer is pancreatic cancer or head and neck cancer (in particular squamous cell carcinoma of the head and neck (SCCHN)), then the method according to the invention may be used to treat or prevent cancer resistance to either tyrosine kinase EGFR inhibitors (such as Erlotinib, Gefitinib, Afatinib Lapatinib or Osimertinib) or to anti-EGFR monoclonal antibodies (such as Cetuximab or Panitumumab).

As used herein, the term “drug resistant” refers to a condition which demonstrates acquired resistance. With “acquired resistance” is meant a multifactorial phenomenon occurring in tumor formation and progression that can influence the sensitivity of cancer cells to a drug. Acquired resistance may be due to several mechanisms such as but not limited to; alterations in drug-targets, decreased drug accumulation, alteration of intracellular drug distribution, reduced drug-target interaction, increased detoxification response, cell-cycle deregulation, increased damaged-DNA repair, and reduced apoptotic response. Several of said mechanisms can occur simultaneously and/or may interact with each other.

Various qualitative and/or quantitative methods may be used to determine if a patient has developed or is susceptible to developing a resistance to treatment with an EGFR TKI such as Gefitinib or Osimertinib or with an EGFR inhibitor that targets the EGFR extracellular domain such as such as Cetuximab or Panitumumab. For example, a patient who showed initial improvement while taking an EGFR inhibitor may display signs that the EGFR inhibitor has become less effective or is no longer effective. Symptoms that may be associated with resistance to an EGFR inhibitor include, for example, a decline or plateau of the well-being of the patient, an increase in the size of a tumor, arrested or slowed decline in growth of a tumor, and/or the spread of cancerous cells in the body from one location to other organs, tissues or cells.

A decrease in the sensitivity of cancer cells to an EGFR inhibitor, an increase in the growth or proliferation of cancer cells, and/or a decrease in cancer cell apoptosis as compared to a control, may also be indicative that the patient has developed or is susceptible to developing a resistance to an EGFR inhibitor. It is possible to determine cancer cell sensitivity, growth, proliferation or apoptosis using standard methods as described further herein. For example, cancer cell sensitivity, growth, proliferation or apoptosis may be determined either in situ or in vitro.

In situ measurements may involve, for example, observing the effect of an EGFR inhibitor therapy in a patient by examining cancer growth or metastasis. Typically, for cancer patients, RECIST criteria are analyzed.

As used herein, the term “Response Evaluation Criteria In Solid Tumors (RECIST)” refers to a set of published rules that define when cancer patients improve (“respond”), stay the same (“stable”) or worsen (“progression”) during treatments. The original criteria were published in February 2000 by an international collaboration including the European Organization for Research and Treatment of Cancer (EORTC), National Cancer Institute (NCI) of the United States and the National Cancer Institute of Canada Clinical Trials Group. RECIST 1.1, published in January 2009, is an update to the original criteria. Usually, the skilled in the art concludes that the disease progresses (and hence that the patient is or is become resistant to a treatment) when at least a 20% increase in the sum of the longest diameter of target lesions, taking as reference the smallest sum longest diameter recorder since the treatment started or the appearance of one or more new lesions) by conventional methods of imaging such as computed tomography (CT).

Within the context of the invention, and according to the RECIST criteria applied to cancer patients, a patient is considered as resistant when at least a 30% increase of metastases is detected in said patient by [¹⁸F]fluoro-2-deoxy-2-d-glucose (FUG) positron emission tomography (PET) imaging (FDG-PET scan).

In one embodiment of the invention, the patient with an acquired resistance is still under EGFR inhibitor treatment.

In another aspect, the invention relates to a method for treating cancer in a patient with an acquired resistance to treatment with an epidermal growth factor receptor (EGFR) inhibitor comprising the following steps of: a) selecting a patient with cancer who has developed a resistance to treatment with an EGFR inhibitor and b) administering to said patient an therapeutically effective amount of a sorafenib drug or sorafenib analogue.

By “therapeutically effective amount” is meant an amount sufficient to achieve a concentration of compound which is capable of preventing or slowing down the disease to be treated. Such concentrations can be routinely determined by those of skilled in the art. The amount of the polypeptide actually administered will typically be determined by a physician or a veterinarian, in the light of the relevant circumstances, including the condition to be treated, the chosen route of administration, the actual compound administered, the age, weight, and response of the patient, the severity of the subject's symptoms, and the like. It will also be appreciated by those of skilled in the art that the dosage may be dependent on the stability of the administered compound.

The compounds of the invention may be administered by any means that achieve the intended purpose. For example, administration may be achieved by a number of different routes including, but not limited to, subcutaneous, intravenous or parenteral, intramuscular, intraperitoneal or oral routes. The parenteral route is particularly preferred for an inhibitor that targets the extracellular domain of EGFR, such as Cetuximab or Panitumumab. The oral route is particularly preferred for sorafenib and EGFR tyrosine kinase inhibitors (TKI), such as Erlotinib, Gefitinib, Lapatinib, Afatinib or Osimertinib.

Dosages to be administered depend on individual needs, on the desired effect and the chosen route of administration. It is understood that the dosage administered will be dependent upon the age, sex, health, and weight of the recipient, concurrent treatment, if any, frequency of treatment, and the nature of the effect desired. The total dose required for each treatment may be administered by multiple doses or in a single dose.

The doses used for the administration can be adapted as a function of various parameters, and in particular as a function of the mode of administration used, of the relevant pathology, or alternatively of the desired duration of treatment. For example, it is well within the skill of the art to start doses of the compounds at levels lower than those required to achieve the desired therapeutic effect and to gradually increase the dosage until the desired effect is achieved. However, the daily dosage of the polypeptides may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 10 mg/kg of body weight per day. Typically in a cancer therapy, patient is treated with 400 mg of sorafenib two times per day or with 80 mg of osimertinib per day.

In a second aspect, the invention relates to a method of preventing and/or treating cancer acquired resistance to treatment with an epidermal growth factor receptor (EGFR) inhibitor in a subject in need thereof comprising administering to the subject a combination of drugs selected from the group consisting of a sorafenib drug or sorafenib analogue and an EGFR inhibitor.

In a third aspect, the invention relates to a method of preventing emergence of resistance to treatment with an epidermal growth factor receptor (EGFR) inhibitor in a subject in need thereof comprising administering to the subject a combination of sorafenib drug or a sorafenib analogue together with a EGFR inhibitor

The term “prevention of resistance” refers to an approach aimed at blocking or delaying the amplification and propagation of EGFR inhibitor resistant cells within the tumor. Such resistant cells may be already present before the onset of the treatment with EGFR inhibitors, or they may result from de novo acquisition of resistance during treatment with EGFR inhibitors (Hata et al., Nat Med 2016; 22: 262-269).

As used herein, the terms “combination” refers to a “kit-of-parts” in the sense that the combination partners as defined above can be dosed independently or by use of different fixed combinations with distinguished amounts of the combination partners, i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partners to be administered in the combined preparation can vary. The combination partners can be administered by the same route or by different routes. When the administration is sequential, the first partner may be for instance administered 1, 2, 3, 4, 5, 6, 7, days before the second partner.

The invention will be further illustrated by the following figures and examples. However, these examples and figures should not be interpreted in any way as limiting the scope of the present invention.

### FIGURES

FIG. 1: Sorafenib prevents selection of EGFR TKI resistant NSCLC cells. (A) PC9 cells containing EGFR-T790 CRISPR-barcodes were treated for 5 days in the presence of the indicated compounds, followed by genomic DNA (gDNA) extraction. The fraction of the EGFR-T790T and EGFR-T790M barcodes was assessed by qPCR and normalized to the total amount of gDNA using EGFR_Ctrl primers. *p<0.05 compared to control (Mann-Whitney test). (B) Colony forming assay using the cells described in A, treated in the presence of the indicated concentrations of gefitinib and sorafenib for 14 or 22 days.

FIG. 2: Effects of sorafenib on EGFR TKI resistance in other NSCLC cells. HCC287 and HCC4006 NSCLC cells transfected for EGFR-T790 CRISPR-barcoding were treated in the presence or the absence of gefitinib (HCC827 cells: 50 nM; HCC4006 cells: 1 μM) and sorafenib (5 μM) for five (HCC827 cells) or nine (HCC4006) days, and the fraction of the EGFR-T790M barcode was measured by qPCR. The mean values (±SEM; n≥4) of one representative of three independent experiments were normalized to the total amount of gDNA using EGFR_Ctrl primers. *p<0.05 and **p<0.01 compared to control (Mann-Whitney test).

FIG. 3: Sorafenib prevents NSCLC resistance to osimertinib induced by the EGFR-C797S mutation. (A) EGFR-T790M containing PC9 cells were selected for 2-3 weeks in the presence of gefitinib (1 μM), followed by CRISPR-barcoding transfection to generate a small subpopulation of cells containing the EGFR-C797S mutation, conferring resistance to third generation EGFR TKI. Cells were then treated for nine days in the presence or the absence of osimertinib (1 μM) and sorafenib (5 μM), and the proportion of the EGFR-C797S barcode was analyzed by qPCR from gDNA. The mean values (±SEM; n=4) of one representative of three independent experiments were normalized to the total amount of gDNA using EGFR_Ctrl primers. *p<0.05 compared to control (Mann-Whitney test). (B) Colony forming assay using the cells described in A, treated for 14 days in the presence of osimertinib (1 μM), with or without sorafenib (5 μM)

FIG. 4: Early effects of sorafenib on STAT3, but not MAPK in NSCLC cells. PC9 cells were treated with sorafenib (5 μM) or the MEK inhibitor trametinib (50 nM) and cell lysate was derived after 2 or 6 hours. (B) Parental and gefitinib resistant (EGFR-T790M) PC9 cells were treated for two days in the presence of sorafenib (5 μM) or gefitinib (1 μM). Immunoblot was performed using the indicated antibodies.

FIG. 5: Long-term sorafenib treatment induces EGFR down-regulation in NSCLC cells. Parental (A) or EGFR-T790M (B) PC9 cells were treated for three days in the presence or the absence of gefitinib (1 μM) and sorafenib (5 μM), and immunoblot was performed using the indicated antibodies.

FIG. 6: Effects of sorafenib in other NSCLC cells. HCC827 and HCC4006 cells were treated for three or five days with sorafenib (5 μM) and immunoblot was performed using the indicated antibodies

FIG. 7: Lack of synergy between sorafenib and EGFR TKI. (A) EGFR-T790M (osimertinib sensitive) and EGFR-T790M/C797S (osimertinib resistant) PC9 cells were treated with or without sorafenib (5 μM) or osimertinib (1 μM) for four days, followed by immunoblot using the indicated antibodies. (B) Colony forming assay using parental, EGFR-T790M and EGFR-T790M/C797S PC9 cells treated for five days with sorafenib (5 μM), osimertinib (1 μM) or a combination of the two drugs.

FIG. 8: Lack of synergy between sorafenib and EGFR TKI. Parental and gefitinib-resistant EGFR-T790M PC9 cells were treated with or without sorafenib (5 μM) or gefitinib (1 μM) for three days, followed by immunoblot. Note that after three days of treatment gefitinib was no more able to inhibit EGFR phosphorylation. Lower panel, colony forming assay to assess the effects of these treatments (four days) on cell growth

FIG. 9: Sorafenib prevents NSCLC cell resistance to EGFR TKI in vivo. CRISPR-barcoding EGFR-C797S PC9 cells described in FIG. 3 were bilaterally inoculated in the flanks of male SCID mice (2×10⁶ cells per site with matrigel) and the volume of the tumors was measured by caliper. At day 27 (arrow), the mice were treated five days a week with osimertinib (5 mg/kg), sorafenib (60 mg/kg), or a combination of the two drugs. From day 39, the mice were treated three times per week. The mean tumor size ±SEM is represented for the different groups (5 mice per group).

FIG. 10: Selection of EGFR-C797S cells in the presence of osimertinib. (A) The fraction of the EGFR-C797S barcode in each tumor from FIG. 9 was assessed by qPCR and normalized to the total amount of gDNA. The proportions of the barcode in the cells prior to mouse injection was analyzed in parallel and arbitrarily set to 1. (B) For each group, the mean of the values (±SEM) shown in A is represented.

FIG. 11: SC-1 prevents resistance of NSCLC cells to EGFR TKI. (A) PC9 cells containing the EGFR-T790 CRISPR-barcodes were treated for 5 days in the presence of gefitinib (gef.; 1 μM) alone or in combination with sorafenib (sor.; 5 μM) or SC-1 (20 μM), followed by genomic DNA (gDNA) extraction. The fraction of the EGFR-T790T and EGFR-T790M barcodes was assessed by qPCR and normalized to the total amount of gDNA using EGFR_Ctrl primers. The mean values (±SEM) representative of four independent experiments are represented. (B). PC9 cells were treated for 3 days in the presence or the absence of sorafenib (5 μM) or SC-1 (20 μM), followed by immunoblot using the indicated antibodies.

FIG. 12: Sorafenib prevents NSCLC resistance to osimertinib in the absence of the EGFR-T790M mutation. Parental PC9 cells (EGFR-T790 wild-type) were transfected to generate through CRISPR-barcoding a small subpopulation of cells containing the EGFR-C797S mutation. Cells were then treated for nine days in the presence or the absence of osimertinib (1 μM) and sorafenib (5 μM), and the proportion of the EGFR-C797S barcode was analyzed by qPCR from gDNA. The mean values (±SEM; n=4) of one representative of three independent experiments were normalized to the total amount of gDNA using EGFR_Ctrl primers.

FIG. 13: Sorafenib prevents cetuximab resistance of colon cancer cells induced by the EGFR-G465R mutation. LIM1215 colon cancer cells were transfected to generate through CRISPR-barcoding a small subpopulation of cells containing the EGFR-G465R mutation. The cells were then treated for six days in the presence or the absence of cetuximab (20 μg/ml) alone or in combination with sorafenib (1 or 5 μM), and the proportion of the EGFR-G465R barcode was analyzed by qPCR from gDNA. The mean values (±SEM; n=3) of one representative of two independent experiments were normalized to the total amount of gDNA using EGFR_Ctrl primers.

### EXAMPLE 1

Material & Methods

Cell Culture, Transfection and Inhibitors

NSCLC cells (PC9, HCC827 and HCC4006) were grown in Roswell Park Memorial Institute medium (Life technologies), supplemented with 10% fetal bovine serum (Life Technologies) and 0.5% penicillin/streptomycin (Life technologies). Cells were transfected with a Nucleofector II device (Lonza) using the Amaxa Nucleofector kit (Lonza) and electroporation program recommended by the manufacturer. Gefitinib, sorafenib and trametinib were purchased from Santa-Cruz Biotechnology. Osimertinib was purchased from MedChemexpress.

CRISPR Barcoding

sgRNA target sequences (Table 1) were designed using the CRISPR Design tool hosted by the Massachusetts Institute of Technology (http://crispr.mit.edu) to minimize potential off-target effects. Oligos encoding the targeting sequence were then annealed and ligated into the pSpCas9(BB)-2A-Puro (Ran et al., 2013) vector digested with BbsI (New England Biolabs). The sequence of the ssODNs (Integrated DNA Technologies) used for CRISPR/Cas9-mediated HDR, containing one missense mutation coupled to different silent mutations, are provided in Table 1. The set of silent mutations is designed to enable PCR specificity and to avoid recognition by the corresponding sgRNA used to cleave the endogenous sequence. For each targeted locus, cells were co-transfected with 2 μg of the CRISPR/Cas9 plasmid and 2 μL of either the control or the sense/nonsense ssODN (50 μM) to prevent the potential incorporation of the two donor DNA sequences into different alleles within the same cell. Immediately after transfection, the cells were pooled in the same flask.

qPCR

gDNA was extracted using the NucleoSpin Tissue kit (Macherey-Nagel). The sequence of the different PCR primers, designed using Primer-BLAST (NCBI), is provided in Table 2. To avoid potential amplification from ssODN molecules not integrated in the correct genomic locus, one of the two primers was designed to target the endogenous genomic sequence flanking the region sharing homology with the ssODNs. Primer specificity for each particular barcode was assessed. qPCR was performed from 100 ng of gDNA using SYBR Green (Life Technologies) on a 7900 HT Fast-Real-Time PCR System (Life Technologies). qPCR analysis was performed using the standard curve method.

Immunoblot

Cells were washed once with phosphate-buffered saline (PBS) and lysed on ice in a buffer containing 50 mM Hepes pH 7.6, 150 mM NaCl, 5 mM EDTA, 1% Nonidet P-40, 20 mM NAF, 2 mM sodium orthovanadate, supplemented with protease inhibitor mini tablets (Thermo Scientific). Lysates were cleared by centrifugation at 14,000 g during 15 min at 4° C. and protein concentrations were determined by using the Bradford protein assay (Bio-Rad). Sodium dodecyl sulfate (SDS) loading buffer was added to equal amounts of lysate, followed by SDS-polyacrylamide gel electrophoresis (SDS-PAGE) and transfer to polyvinylidene fluoride (PVDF) membrane (Thermo Scientific). Membranes were analyzed by chemiluminescence using a ChemiDoc imaging system (Biorad). All primary antibodies were purchased from Cell Signaling. Secondary antibodies were purchased from Santa Cruz.

Mouse Xenografts

Gefitinib resistant PC9 cells (EGFR-T790M) containing the EGFR-C797 CRISPR-barcodes were mixed with matrigel and subcutaneously inoculated in the left and right flank (2×106 cells per site) of male SCID mice. The size of the tumors was measured by caliper every 3-4 days. 27 days after injection, when the tumors reached a mean volume of 40-50 mm3, the mice were treated five days a week by gavage with osimertinib (5 mg/kg), sorafenib (60 mg/kg) or a combination of the two drugs. From day 39, the mice were treated three times per week. Tumors from sacrificed mice were dissected, and gDNA was derived using the DNeasy Blood & Tissue Kit (Qiagen). All animal experiments were approved and performed according to the relevant regulatory standards set by Mount Sinai's Animal Care and Use Committee.

Results

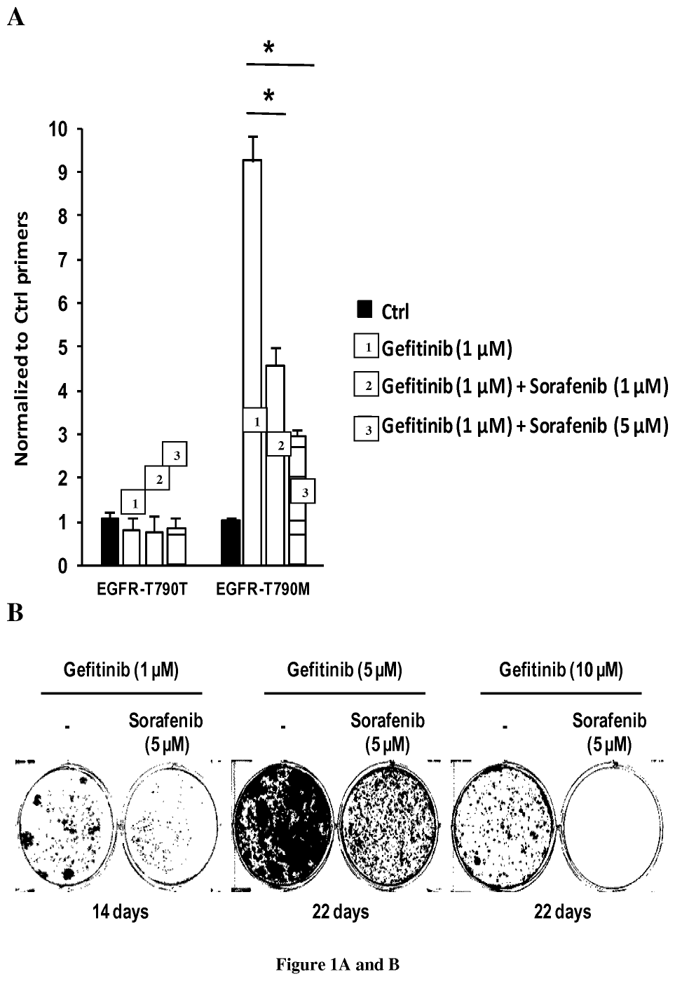

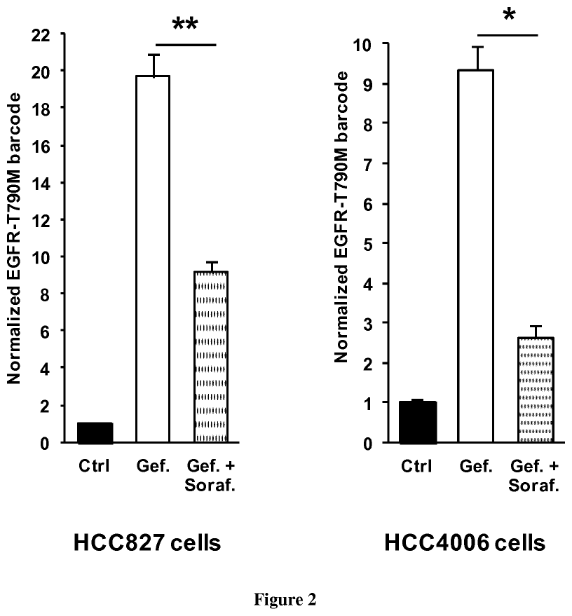

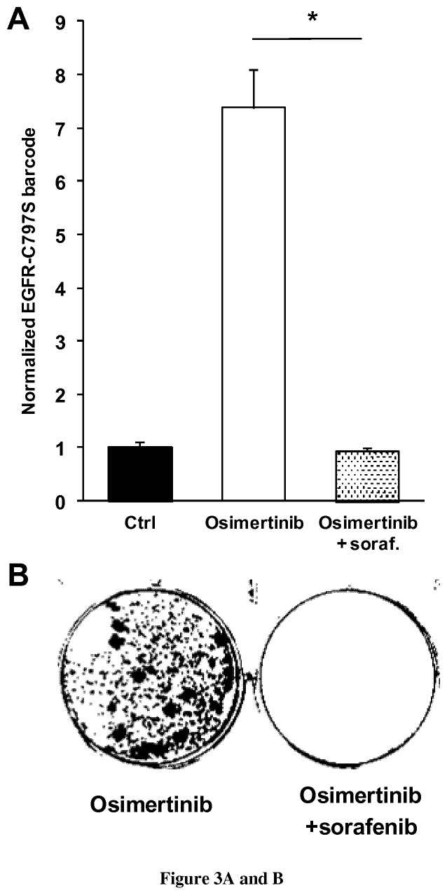

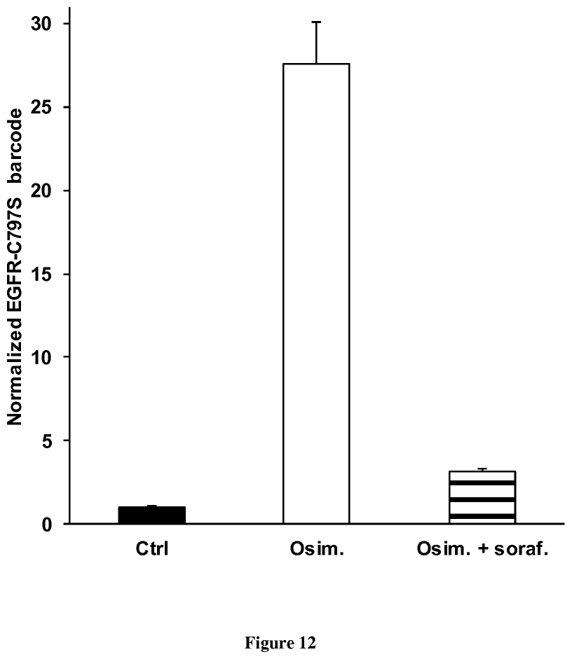

Taking advantage of our CRISPR-barcoding approach (Guernet et al., 2016), we recently performed a screen for small molecules potentially capable of inhibiting NSCLC resistance to EGFR TKI. Among the different compounds tested, the multikinase inhibitor sorafenib dose-dependently prevented the enrichment of PC9 cells containing the EGFR-T790M mutation in the presence of gefitinib (FIG. 1A), as measured by qPCR from genomic DNA (gDNA). Consistent with these data, co-treatment with sorafenib abolished the formation of resistant colonies in the presence of gefitinib (FIG. 1B). Similar results were obtained in other EGFR TKI-sensitive NSCLC cells, including HCC827 and HCC4006 (FIG. 2). To investigate the effects of sorafenib on NSCLC resistance to third generation EGFR TKI, we generated through CRISPR-barcoding a small subset of clones containing the EGFR-C797S mutation within a mass population of PC9-(EGFR-T790M) cells previously selected in the presence of gefitinib. As measured by qPCR (FIG. 3A) or colony forming assay (FIG. 3B), treatment with osimertinib induced a strong amplification of EGFR-C797S cells, which was blocked by co-treatment with sorafenib. In recent clinical trials, first-line treatment with osimertinib has shown superior efficacy and less severe adverse effects compared to first/second generation TKIs (Soria et al., N Engl J Med 2018; 378:113-125). As illustrated in FIG. 12, sorafenib strongly inhibited the enrichment of EGFR-C797S cells induced by osimertinib in PC9 cells that were not previously selected in the presence of other EGFR TKIs. Together, these data indicate that sorafenib can also prevent NSCLC resistance to latest generation EGFR TKI, regardless of the EGFR-T790 status.

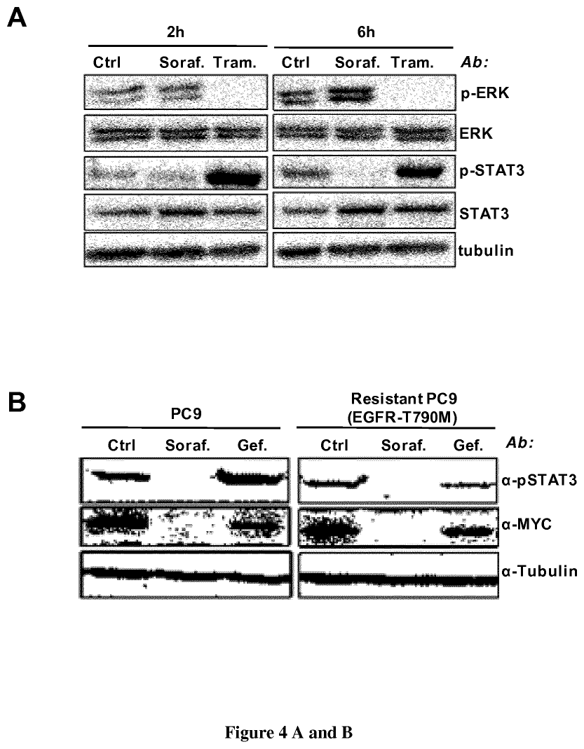

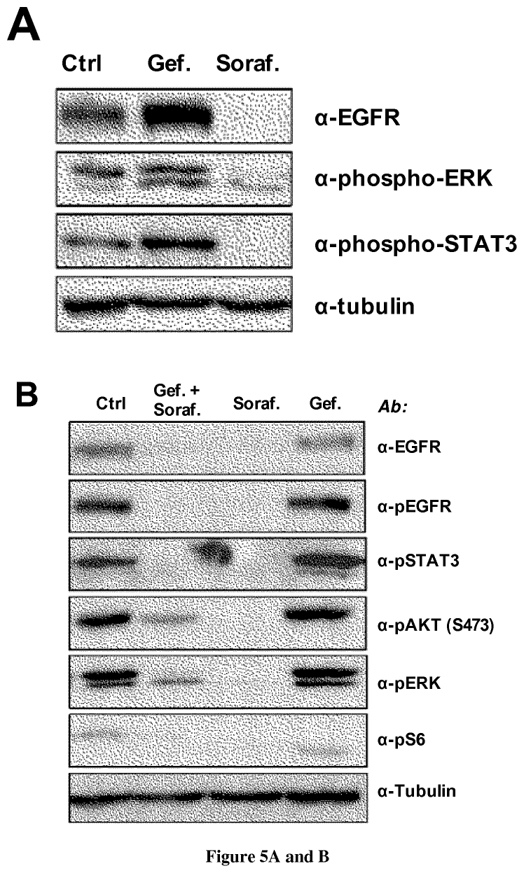

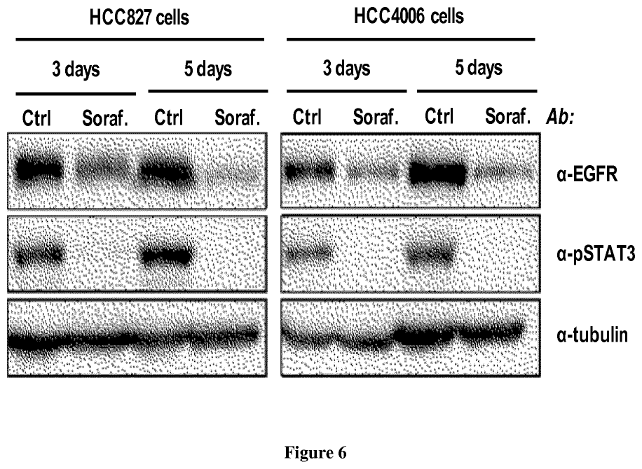

Approved by the FDA for the treatment of advanced renal cell carcinoma in 2005, for hepatocellular carcinoma in 2007 and for thyroid carcinoma in 2013, sorafenib inhibits several tyrosine kinases, including VEGFR, PDGFR, c-Kit and Ret, as well as the serine/threonine kinase RAF (Wilhelm et al., 2006). Given the well-established role of VEGFR and PDGFR on the development and maintenance of blood and lymphatic vessels, sorafenib is often considered as an inhibitor of tumor angiogenesis, although the molecular mechanisms responsible for its activity against certain types of cancer have not been fully characterized. A systematic investigation of EGFR signaling in different EGFR TKI sensitive NSCLC cells revealed that sorafenib can induce a rapid inhibition of STAT3 phosphorylation (FIG. 4). Of note, despite its reported anti-RAF activity, this compound had no effect on MAPK activation (FIG. 4A), but it down-regulated expression of MYC transcription factor (FIG. 4B). After three days of sorafenib treatment, we observed a dramatic decrease of EGFR expression in PC9 cells, which was accompanied by inhibition of different downstream pathways regulated by this receptor, including MAPK, AKT and mTOR (FIG. 5). Similar results were obtained in other NSCLC cells (FIG. 6). Together, these data indicate that sorafenib can prevent EGFR TKI resistance through a dual mechanism that involves early inhibition of STAT3 and later down-regulation of MYC and EGFR signaling.

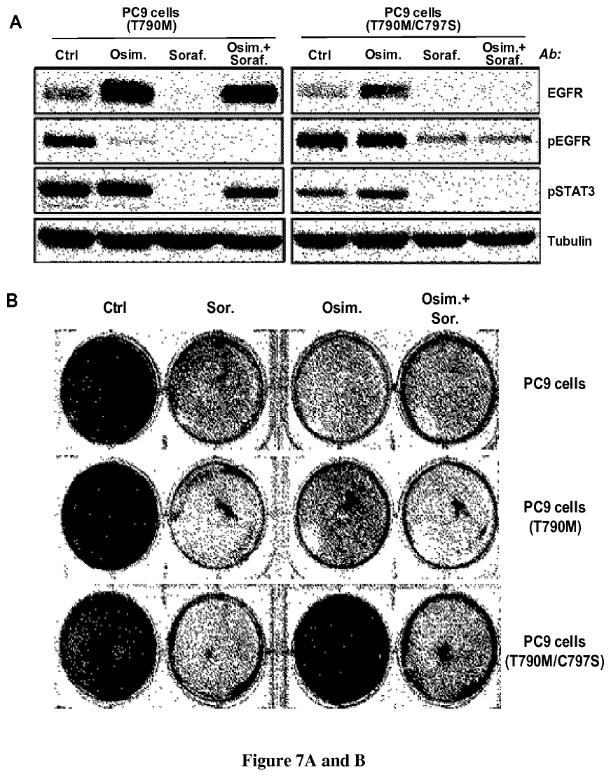

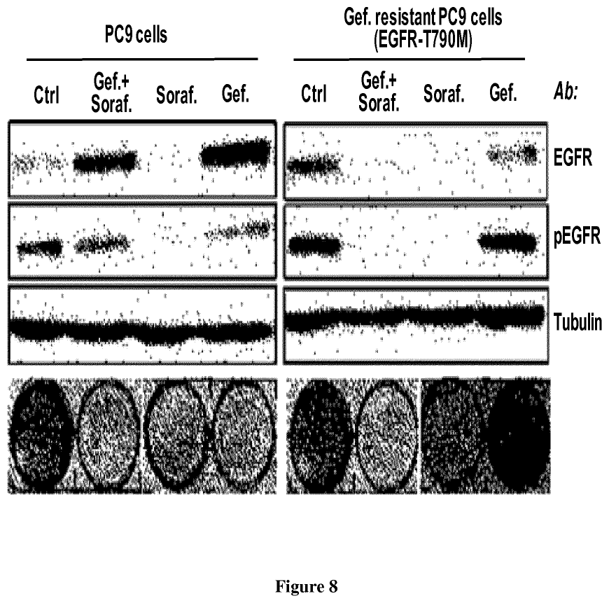

Our CRISPR-barcoding experiments revealed that sorafenib was able to block selection of resistant cells in the presence of EGFR TKI, suggesting that these cells may display a greater vulnerability to this compound. To test this hypothesis, we compared the effects of osimertinib and sorafenib in osimertinib resistant (EGFR-T790M/C797S) and sensitive (EGFR-T790M) PC9 cells. As shown in FIG. 7A, combination of these two drugs induced EGFR down-regulation and phospho-STAT3 inhibition in PC9 cells containing the EGFR-T790M/C797S mutations, but not in osimertinib sensitive cells. Consistent with these findings, both sorafenib and osimertinib inhibited cell growth as single agents, but did not exert a synergistic effect in combination (FIG. 7B). Similar results were obtained for the combination of gefitinib and sorafenib in parental and gefitinib-resistant (EGFR-T790M) PC9 cells (FIG. 8).

Before the onset of resistance, targeted therapies induce a dramatic decrease in tumor heterogeneity, through generation of a sort of evolutionary bottleneck that constitutes a potential window of opportunity for drug combinations (Gerlinger and Swanton, 2010; Merlo et al., 2006). Our studies established that sorafenib, in combination with EGFR TKI, specifically prevents selection of resistant clones bearing secondary/tertiary EGFR mutations, through an unusual mechanism, which involves specific targeting of cells unresponsive to EGFR TKI. This effect, which could probably not have been identified through more conventional approaches, also implies that emergent clones that acquire resistance to EGFR TKI would be immediately exposed to a new type of selective pressure, from which they were previously sheltered because of their responsiveness to EGFR TKI. Thus, compared to agents that exert a synergistic/additive effect with EGFR TKI, sorafenib co-treatment could provide a more efficient and potentially less toxic strategy to prevent amplification of resistant cells.

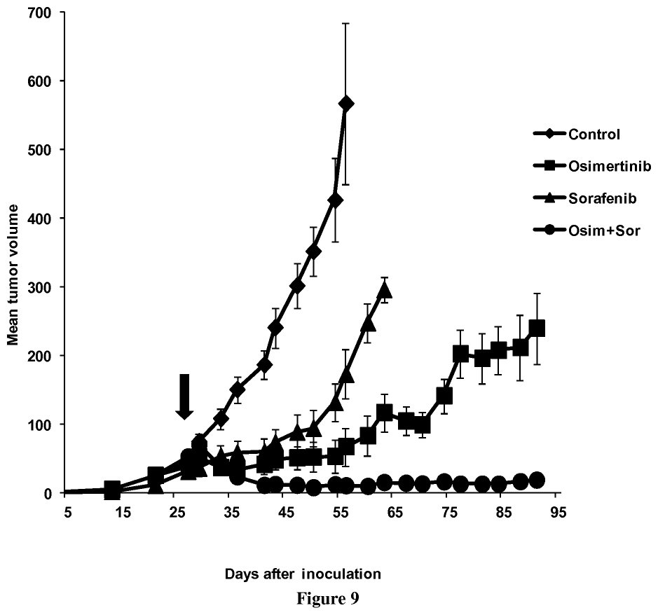

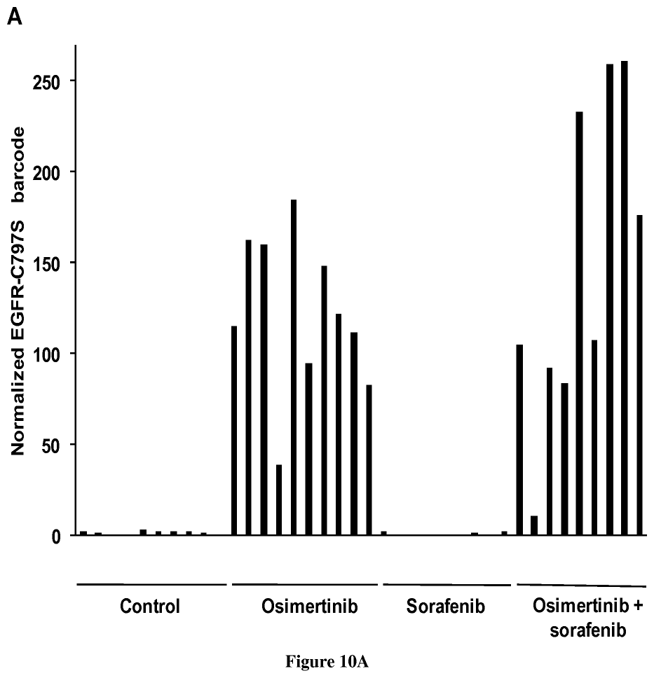

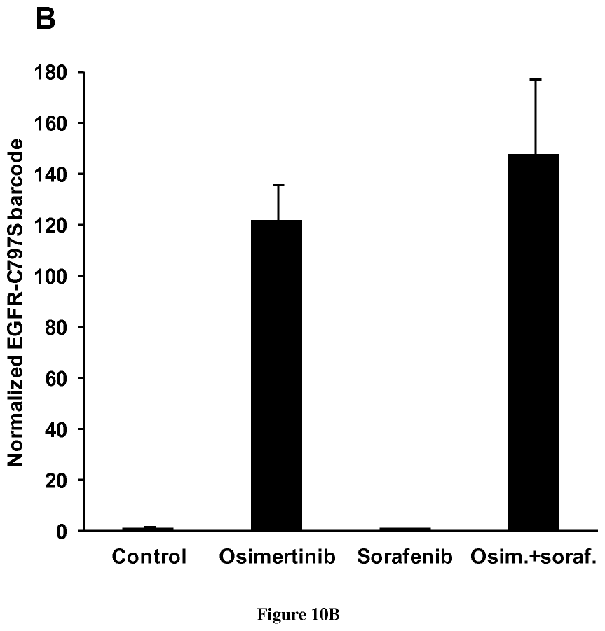

To confirm our in vitro data, EGFR-T790M PC9 cells containing the EGFR-C797S CRISPR-barcode described in FIG. 3 were subcutaneously and bilaterally injected in immunocompromised mice. Four weeks after inoculation, when the volume of the tumors reached 40-50 mm3, the mice were treated in the presence or the absence of osimertinib (5 mg/kg); sorafenib (60 mg/kg) or a combination of the two drugs. As illustrated in FIG. 9, in the control group the tumors grew rapidly, and all the mice were sacrificed three weeks later. In the presence of sorafenib or osimertinib, the growth of the tumors was initially inhibited, but it eventually resumed after a few weeks. Conversely, combination of these two drugs provoked a decrease of tumor size, which remained stable for two months, when the remaining mice were sacrificed. These results are consistent with our previous data and indicate that sorafenib co-treatment can prevent NSCLC cell resistance to EGFR TKI in vivo. gDNA was derived from the different tumors and we performed qPCR to assess the proportion of barcoded cells. FIG. 10 shows that EGFR-C797S cells were enriched more than 100 fold in both osimertinib and osimertinib+sorafenib groups. Considering that the efficiency of CRISPR-barcoding in PC9 cells is about 0.5-1% (Guernet et al., 2016), these data indicate that osimertinib treatment eliminated most, if not all, sensitive cells, while the growth of EGFR-C797S resistant cells was arrested by sorafenib.

### TABLES

TABLE 1
related to the Experimental Procedures. List of sgRNA
target sequences and ssODNs used for CRISPR-barcoding.
sgRNA target

| Name | Sequence 5′-3′ |
|---|---|
| sgEGFR-T790 | CTGCGTGATGAGCTGCACGG (SEQ ID No 1) |
| sgEGFR-C797 | CATGCCCTTCGGCTGCCTCC (SEQ ID No 2) |
| sgEGFR-G465 | TCCCTCAAGGAGATAAGTGA (SEQ ID No 15) |

ssODN

| Name | Sequence 5′-3′ (a) |
|---|---|
| EGFR-T790T | CTCCCTCCCTCCAGGAAGCCTACGTGATGGCCAGCGTGGA |
| | CAACCCCCACGTGTGCCGCCTGCTGGGCATCTGCCTCACCT |
| | CtACaGTcCAaCTgATtACcCAGCTCATGCCCTTCGGCTGCCTC |
| | CTGGACTATGTCCGGGAACACAAAGACAATATTGGCTCCC |
| | AGTACCTGCTCAACTGGTGTGTG (SEQ ID No 3) |
| EGFR-T790M | CTCCCTCCCTCCAGGAAGCCTACGTGATGGCCAGCGTGGA |
| | CAACCCCCACGTGTGCCGCCTGCTGGGCATCTGCCTCACCT |
| | CtACtGTaCAGCTtATaAtGCAaCTgATGCCCTTCGGCTGCCTCC |
| | TGGACTATGTCCGGGAACACAAAGACAATATTGGCTCCCA |
| | GTACCTGCTCAACTGGTGTGTG (SEQ ID No 4) |
| EGFR-C797C | GATGGCCAGCGTGGACAACCCCCACGTGTGCCGCCTGCTG |
| | GGCATCTGCCTCACCTCCACCGTGCAGCTCATCAtGCAGCT |
| | CATGCCgTTtGGtTGtCTaCTcGACTATGTCCGGGAACACAAA |
| | GACAATATTGGCTCCCAGTACCTGCTCAACTGGTGTGTGCA |
| | GATCGCAAAGGTAATCAGGGAAG (SEQ ID No 5) |
| EGFR-C797S | GATGGCCAGCGTGGACAACCCCCACGTGTGCCGCCTGCTG |
| | GGCATCTGCCTCACCTCCACCGTGCAGCTCATCAtGCAGCT |
| | CATGCCCTTCGGgaGtCTgCTtGAtTAcGTCCGGGAACACAAA |
| | GACAATATTGGCTCCCAGTACCTGCTCAACTGGTGTGTGCA |
| | GATCGCAAAGGTAATCAGGGAAG (SEQ ID No 6) |
| EGFR- | GATGGCCAGCGTGGACAACCCCCACGTGTGCCGCCTGCTG |
| C7975_(T790_wt) | GGCATCTGCCTCACCTCCACCGTGCAGCTCATCACGCAGCT |
| | CATGCCCTTCGGgaGtCTgCTtGAtTAcGTCCGGGAACACAAA |
| | GACAATATTGGCTCCCAGTACCTGCTCAACTGGTGTGTGCA |
| | GATCGCAAAGGTAATCAGGGAAG (SEQ ID No 16) |
| EGFR- | GATGGCCAGCGTGGACAACCCCCACGTGTGCCGCCTGCTG |
| C797C_(T790_wt) | GGCATCTGCCTCACCTCCACCGTGCAGCTCATCACGCAGCT |
| | CATGCCgTTtGGtTGtCTaCTcGACTATGTCCGGGAACACAAA |
| | GACAATATTGGCTCCCAGTACCTGCTCAACTGGTGTGTGCA |
| | GATCGCAAAGGTAATCAGGGAAG (SEQ ID No 17) |
| EGFR-G465R | TTTCTTCTCTCCAATGTAGTGGTCAGTTTTCTCTTGCAGTCG |
| | TCAGCCTGAACATAACATCCTTGGGATTACGCTCCCTCAAG |
| | GAaATtAGcGATGGtGAcGTcATtATcTCAaGAAACAAAAATTT |
| | GTGCTATGCAAATACAATAAACTGGAAgAAACTGTTTGGG |
| | ACCTCCGGTCAGAAAACCAAAATTA (SEQ ID No 18) |
| EGFR-G465G | TTTCTTCTCTCCAATGTAGTGGTCAGTTTTCTCTTGCAGTCG |
| | TCAGCCTGAACATAACATCCTTGGGATTACGCTCCCTCAAG |
| | GAaATcAGcGATGGcGATGTtATcATaTCAGGcAACAAAAATT |
| | TGTGCTATGCAAATACAATAAACTGGAAgAAACTGTTTGG |
| | GACCTCCGGTCAGAAAACCAAAATTA (SEQ ID No 19) |

(a) Mutations compared to the endogenous sequence are indicated in lowercase letters

TABLE 2
List of primers used for qPCR.
qPCR primers

| Name | Sequence 5'-3' |
|---|---|
| EGFR_Ctrl_FW | TGCTTCCCCCATTCAGGACT |
| | (SEQ ID No 7) |
| EGFR_Ctrl_RV | CTCCTTGCACCTCCTCACTG |
| | (SEQ ID No 8) |
| EGFR_cbc_T790_RV | CCTTCCCTGATTACCTTTGCGA |
| | (SEQ ID No 9) |
| EGFR_cbc_T790T_FW | CACCTCTACAGTCCAACTGATTACC |
| | (SEQ ID No 10) |
| EGFR_cbc_T790M_FW | CTCTACTGTACAGCTTATAATGCAACTG |
| | (SEQ ID No 11) |
| EGFR_cbc_C797_RV | CCTTATCTCCCCTCCCCGTAT |
| | (SEQ ID No 12) |
| EGFR_cbc_C797C_FW | CATGCCGTTTGGTTGTCTACTC |
| | (SEQ ID No 13) |
| EGFR_cbc_C797S_FW | CTTCGGGAGTCTGCTTGATTAC |
| | (SEQ ID No 14) |
| EGFR_cbc_G465R_FW | CGATGGTGACGTCATTATCTCAA |
| | (SEQ ID No 20) |
| EGFR_cbc_G465_RV | ACTAAACAGAAAGCGGTGACT |
| | (SEQ ID No 21) |

### EXAMPLE 2

Material & Methods

Cell Culture, Transfection and Inhibitors

PC9 cells were grown in Roswell Park Memorial Institute medium (Life technologies), supplemented with 10% fetal bovine serum (Life Technologies) and 0.5% penicillin/streptomycin (Life technologies). Cells were transfected with a Nucleofector II device (Lonza) using the Amaxa Nucleofector kit (Lonza) and electroporation program recommended by the manufacturer. Gefitinib, sorafenib and trametinib were purchased from Santa-Cruz Biotechnology. SC-1 was purchased from Sigma.

CRISPR Barcoding

sgRNA target sequences (example 1, Table 1) were designed using the CRISPR Design tool hosted by the Massachusetts Institute of Technology (http://crispr.mit.edu) to minimize potential off-target effects. Oligos encoding the targeting sequence were then annealed and ligated into the pSpCas9(BB)-2A-Puro (Ran et al., 2013) vector digested with BbsI (New England Biolabs). The sequence of the ssODNs (Integrated DNA Technologies) used for CRISPR/Cas9-mediated HDR, containing one missense mutation coupled to different silent mutations, are provided in Table 1. The set of silent mutations is designed to enable PCR specificity and to avoid recognition by the corresponding sgRNA used to cleave the endogenous sequence. For each targeted locus, cells were co-transfected with 2 μg of the CRISPR/Cas9 plasmid and 2 μL of either the control or the sense/nonsense ssODN (50 μM) to prevent the potential incorporation of the two donor DNA sequences into different alleles within the same cell. Immediately after transfection, the cells were pooled in the same flask.

qPCR

gDNA was extracted using the NucleoSpin Tissue kit (Macherey-Nagel). The sequence of the different PCR primers, designed using Primer-BLAST (NCBI), is provided in Table 2 (example 1). To avoid potential amplification from ssODN molecules not integrated in the correct genomic locus, one of the two primers was designed to target the endogenous genomic sequence flanking the region sharing homology with the ssODNs. Primer specificity for each particular barcode was assessed. qPCR was performed from 100 ng of gDNA using SYBR Green (Life Technologies) on a 7900 HT Fast-Real-Time PCR System (Life Technologies). qPCR analysis was performed using the standard curve method.

Immunoblot

Cells were washed once with phosphate-buffered saline (PBS) and lysed on ice in a buffer containing 50 mM Hepes pH 7.6, 150 mM NaCl, 5 mM EDTA, 1% Nonidet P-40, 20 mM NAF, 2 mM sodium orthovanadate, supplemented with protease inhibitor mini tablets (Thermo Scientific). Lysates were cleared by centrifugation at 14,000 g during 15 min at 4° C. and protein concentrations were determined by using the Bradford protein assay (Bio-Rad). Sodium dodecyl sulfate (SDS) loading buffer was added to equal amounts of lysate, followed by SDS-polyacrylamide gel electrophoresis (SDS-PAGE) and transfer to polyvinylidene fluoride (PVDF) membrane (Thermo Scientific). Membranes were analyzed by chemiluminescence using a ChemiDoc imaging system (Biorad). All primary antibodies were purchased from Cell Signaling. Secondary antibodies were purchased from Santa Cruz.

Results

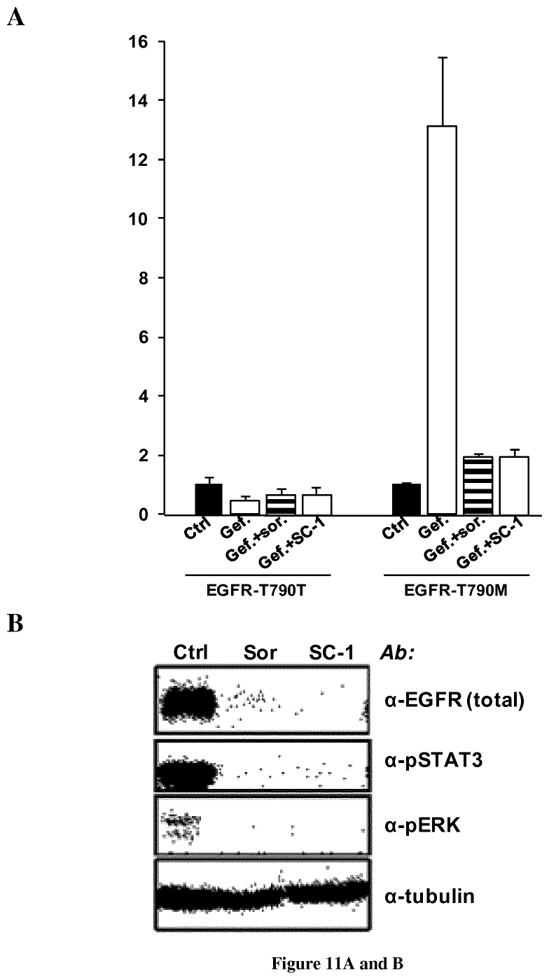

It has been reported that sorafenib analog SC-1 can induce pSTAT3 inhibition and growth inhibition in different cell models, including EGFR-wild type NSCLC cells (Su et al., 2015; Wang et al., 2014). Using our CRISPR-barcoding approach, we found that SC-1 could mimic the effects of sorafenib in preventing the amplification of EGFR-T790M containing cells in the presence of gefitinib (FIG. 11A). Consistent with these results, both sorafenib and SC-1 down-regulated EGFR and inhibited STATS phosphorylation in PC9 cells (FIG. 11B), indicating that these compounds can prevent NSCLC resistance to EGFR TKI through a similar mechanism.

### EXAMPLE 3

Material & Methods

Cell Culture, Transfection and Inhibitors

LIM1215 were grown in RPMI (+25 mM HEPES), supplemented with 10% fetal bovine serum (Life Technologies), Insulin (Sigma) 0.6 μg/ml, Hydrocortisone (Sigma) 1 μg/ml and 1-Thioglycerol (Sigma) 10 μM. Cells were transfected with a Nucleofector II device (Lonza) using the Amaxa Nucleofector kit (Lonza) and electroporation program recommended by the manufacturer. Sorafenib was purchased from Santa-Cruz Biotechnology. Cetuximab was purchased from Selleckchem.

CRISPR Barcoding

sgRNA target sequences (example 1, Table 1) were designed using the CRISPR Design tool hosted by the Massachusetts Institute of Technology (http://crispr.mit.edu) to minimize potential off-target effects. Oligos encoding the targeting sequence were then annealed and ligated into the pSpCas9(BB)-2A-Puro (Ran et al., 2013) vector digested with BbsI (New England Biolabs). The sequence of the ssODNs (Integrated DNA Technologies) used for CRISPR/Cas9-mediated HDR, containing one missense mutation coupled to different silent mutations, are provided in Table 1. The set of silent mutations is designed to enable PCR specificity and to avoid recognition by the corresponding sgRNA used to cleave the endogenous sequence. For each targeted locus, cells were co-transfected with 2 μg of the CRISPR/Cas9 plasmid and 2 μL of either the control or the sense/nonsense ssODN (50 μM) to prevent the potential incorporation of the two donor DNA sequences into different alleles within the same cell. Immediately after transfection, the cells were pooled in the same flask.

qPCR

gDNA was extracted using the NucleoSpin Tissue kit (Macherey-Nagel). The sequence of the different PCR primers, designed using Primer-BLAST (NCBI), is provided in Table 2 (example 1). To avoid potential amplification from ssODN molecules not integrated in the correct genomic locus, one of the two primers was designed to target the endogenous genomic sequence flanking the region sharing homology with the ssODNs. Primer specificity for each particular barcode was assessed. qPCR was performed from 100 ng of gDNA using SYBR Green (Life Technologies) on a 7900 HT Fast-Real-Time PCR System (Life Technologies). qPCR analysis was performed using the standard curve method.

Results

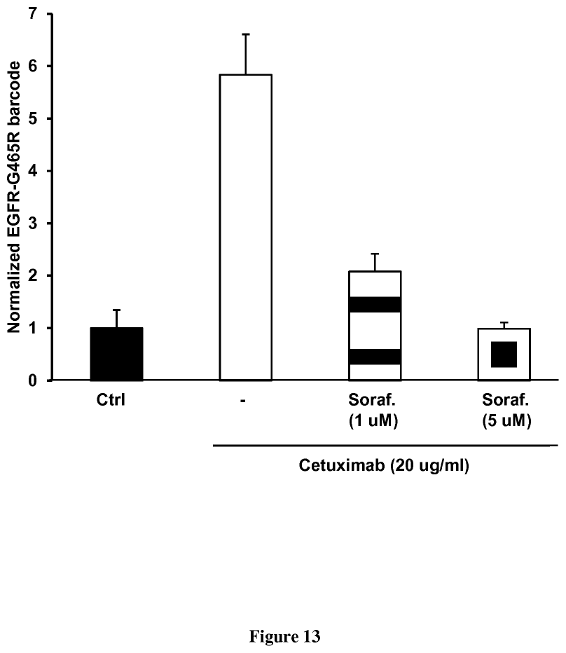

Monoclonal antibodies targeting EGFR, including cetuximab and panitumumab, are used in the treatment of metastatic colorectal cancers displaying wild-type RAS and BRAF. These tumors unfortunately develop secondary resistance to these antibodies, which can be mediated in some cases by mutations in EGFR extracellular domain, including S492R, R451C, S464L, G465R, K467T and I491M (Montagut et al., Nat Med 2012; 18:221-223; Arena et al., Clin Cancer Res 2015; 21:2157-2166). To model this type of acquired resistance, we chose LIM1215 cells, a colon cancer line sensitive to cetuximab (Arena et al., Clin Cancer Res 2015; 21:2157-2166). We applied our CRISPR-barcoding strategy to generate a small subpopulation of cells containing the EGFR-G465R mutation. As shown in FIG. 13, treatment of CRISPR-barcoded LIM1215 cells with cetuximab induced an enrichment of the EGFR-G465R barcode, which was inhibited by sorafenib in a dose-dependent manner. These data indicate that sorafenib can prevent colon cancer cell resistance to anti-EGFR monoclonal antibodies induced by mutations in the extracellular domain of this receptor.

### REFERENCES

Throughout this application, various references describe the state of the art to which this invention pertains. The disclosures of these references are hereby incorporated by reference into the present disclosure.
Arena, S., Bellosillo, B., Siravegna, G., Martinez, A., Canadas, I., Lazzari, L., Ferruz, N., Russo, M., Misale, S., Gonzalez, I., et al. (2015). Emergence of Multiple EGFR Extracellular Mutations during Cetuximab Treatment in Colorectal Cancer. Clin Cancer Res 21, 2157-2166.
Bhang, H. E., Ruddy, D. A., Krishnamurthy Radhakrishna, V., Caushi, J. X., Zhao, R., Hims, M. M., Singh, A. P., Kao, I., Rakiec, D., Shaw, P., et al. (2015). Studying clonal dynamics in response to cancer therapy using high-complexity barcoding. Nat Med 21, 440-448.
Chong, C. R., and Janne, P. A. (2013). The quest to overcome resistance to EGFR-targeted therapies in cancer. Nat Med 19, 1389-1400.
Cortot, A. B., and Janne, P. A. (2014). Molecular mechanisms of resistance in epidermal growth factor receptor-mutant lung adenocarcinomas. European respiratory review: an official journal of the European Respiratory Society 23, 356-366.
Gerlinger, M., and Swanton, C. (2010). How Darwinian models inform therapeutic failure initiated by clonal heterogeneity in cancer medicine. British journal of cancer 103, 1139-1143.
Guernet, A., Mungamuri, S. K., Cartier, D., Sachidanandam, R., Jayaprakash, A., Adriouch, S., Vezain, M., Charbonnier, F., Rohkin, G., Coutant, S., et al. (2016). CRISPR-Barcoding for Intratumor Genetic Heterogeneity Modeling and Functional Analysis of Oncogenic Driver Mutations. Mol Cell 63, 526-538.
Hata, A. N., Niederst, M. J., Archibald, H. L., Gomez-Caraballo, M., Siddiqui, F. M., Mulvey, H. E., Maruvka, Y. E., Ji, F., Bhang, H. E., Krishnamurthy Radhakrishna, V., et al. (2016). Tumor cells can follow distinct evolutionary paths to become resistant to epidermal growth factor receptor inhibition. Nat Med 22, 262-269.
Janne, P. A., Yang, J. C., Kim, D. W., Planchard, D., Ohe, Y., Ramalingam, S. S., Ahn, M. J., Kim, S. W., Su, W. C., Horn, L., et al. (2015). AZD9291 in EGFR inhibitor-resistant non-small-cell lung cancer. N Engl J Med 372, 1689-1699.
Merlo, L. M., Pepper, J. W., Reid, B. J., and Maley, C. C. (2006). Cancer as an evolutionary and ecological process. Nat Rev Cancer 6, 924-935.
Minari, R., Bordi, P., and Tiseo, M. (2016). Third-generation epidermal growth factor receptor-tyrosine kinase inhibitors in T790M-positive non-small cell lung cancer: review on emerged mechanisms of resistance. Translational lung cancer research 5, 695-708.
Montagut, C., Dalmases, A., Bellosillo, B., Crespo, M., Pairet, S., Iglesias, M., Salido, M., Gallen, M., Marsters, S., Tsai, S. P., et al. (2012). Identification of a mutation in the extracellular domain of the Epidermal Growth Factor Receptor conferring cetuximab resistance in colorectal cancer. Nat Med 18, 221-223.
Ran, F. A., Hsu, P. D., Wright, J., Agarwala, V., Scott, D. A., and Zhang, F. (2013). Genome engineering using the CRISPR-Cas9 system. Nature protocols 8, 2281-2308.
Sequist, L. V., Soria, J. C., Goldman, J. W., Wakelee, H. A., Gadgeel, S. M., Varga, A., Papadimitrakopoulou, V., Solomon, B. J., Oxnard, G. R., Dziadziuszko, R., et al. (2015). Rociletinib in EGFR-mutated non-small-cell lung cancer. N Engl J Med 372, 1700-1709.
Soria, J. C., Ohe, Y., Vansteenkiste, J., Reungwetwattana, T., Chewaskulyong, B., Lee, K. H., Dechaphunkul, A., Imamura, F., Nogami, N., Kurata, T., et al. (2018). Osimertinib in Untreated EGFR-Mutated Advanced Non-Small-Cell Lung Cancer. N Engl J Med 378, 113-125.
Thress, K. S., Paweletz, C. P., Felip, E., Cho, B. C., Stetson, D., Dougherty, B., Lai, Z., Markovets, A., Vivancos, A., Kuang, Y., et al. (2015). Acquired EGFR C797S mutation mediates resistance to AZD9291 in non-small cell lung cancer harboring EGFR T790M. Nat Med 21, 560-562.
Tsao, A. S., Scagliotti, G. V., Bunn, P. A., Jr., Carbone, D. P., Warren, G. W., Bai, C., de Koning, H. J., Yousaf-Khan, A. U., McWilliams, A., Tsao, M. S., et al. (2016). Scientific Advances in Lung Cancer 2015. Journal of thoracic oncology: official publication of the International Association for the Study of Lung Cancer 11, 613-638.
Wilhelm, S., Carter, C., Lynch, M., Lowinger, T., Dumas, J., Smith, R. A., Schwartz, B., Simantov, R., and Kelley, S. (2006). Discovery and development of sorafenib: a multikinase inhibitor for treating cancer. Nat Rev Drug Discov 5, 835-844.

1. A method of treating a cancer with an acquired resistance to treatment with an epidermal growth factor receptor (EGFR) inhibitor in a subject in need thereof comprising administering to the subject a sorafenib drug or a sorafenib analogue.

2. The method according to claim 1, wherein the drug is sorafenib.

3. The method according to claim 1, wherein the EGFR inhibitor is an EGFR tyrosine kinase inhibitor (TKI), or an inhibitor which targets the extracellular domain of the EGFR target.

4. The method according to claim 3, wherein the EGFR TKI is selected from the group consisting of Erlotinib, Gefitinib, Lapatinib, Afatinib and Osimertinib.

5. The method according to claim 3, wherein the EGFR inhibitor which targets the extracellular domain of the EGFR target is Cetuximab and Panitumumab.

6. The method according to claim 1, wherein the cancer with an acquired resistance to treatment with an EGFR inhibitor is a colorectal carcinoma cancer or a lung cancer.

7. The method according to claim 6 wherein the lung cancer is a non-small-cell lung cancer (NSCLC).

8. The method according to claim 7, wherein the NSCLC patient has a first activating mutation in EGFR kinase domain which is selected from the group consisting of exon 19 deletions, L858R, exon 20 insertions G719X, L861X and exon 19 insertions.

9. The method according to claim 8, wherein the NSCLC patient has a secondary/tertiary mutation in EGFR kinase domain which is T790M or C797S.

10. The method according to claim 7, wherein the EGFR inhibitor is an EGFR tyrosine kinase inhibitor (TKI) which is selected from the group consisting of Erlotinib, Gefitinib, Lapatinib, Afatinib and Osimertinib.

11. A method for preventing and/or treating cancer in a patient with acquired resistance to treatment with a epidermal growth factor receptor (EGFR) inhibitor comprising administering to the patient a combination of sorafenib drug or a sorafenib analogue and an EGFR inhibitor.

12. A method of preventing emergence of resistance to treatment with an epidermal growth factor receptor (EGFR) inhibitor in a subject in need thereof comprising administering to the subject a combination of sorafenib or a sorafenib analogue, together with a EGFR inhibitor.

13. The method according to claim 11, wherein the patient is treated with sorafenib.

14. The method according to claim 11, wherein the EGFR inhibitor is an EGFR tyrosine kinase inhibitor (TKI), or an inhibitor which targets the extracellular domain of the EGFR target.

15. The method according to claim 11, wherein the cancer with an acquired resistance to treatment with an EGFR inhibitor is a non-small-cell lung cancer (NSCLC).

16. The method according to claim 12, wherein the subject is treated with sorafenib.

17. The method according to claim 12, wherein the EGFR inhibitor is an EGFR tyrosine kinase inhibitor (TKI), or an inhibitor which targets the extracellular domain of the EGFR target.

18. The method according to claim 12, wherein the cancer with an acquired resistance to treatment with an EGFR inhibitor is a non-small-cell lung cancer (NSCLC).


SEQUENCE LISTING (14 sequences)

SEQ ID NO: 1 - DNA, 20 bp, Artificial
  Synthetic sgEGFR-T790
ctgcgtgatgagctgcacgg

SEQ ID NO: 2 - DNA, 20 bp, Artificial
  Synthetic sgEGFR-C797
catgcccttcggctgcctcc

SEQ ID NO: 3 - DNA, 188 bp, Artificial
  Synthetic ssODN EGFR-T790T
ctccctccctccaggaagcctacgtgatggccagcgtggacaacccccacgtgtgccgcctgctgggcatctgcctcacctctacagtccaactgattacccagctcatgcccttcggctgcctcctggactatgtccgggaacacaaagacaatattggctcccagtacctgctcaactggtgtgtg

SEQ ID NO: 4 - DNA, 188 bp, Artificial
  Synthetic ssODN EGFR-T790M
ctccctccctccaggaagcctacgtgatggccagcgtggacaacccccacgtgtgccgcctgctgggcatctgcctcacctctactgtacagcttataatgcaactgatgcccttcggctgcctcctggactatgtccgggaacacaaagacaatattggctcccagtacctgctcaactggtgtgtg

SEQ ID NO: 5 - DNA, 188 bp, Artificial
  Synthetic ssODN EGFR-C797C
gatggccagcgtggacaacccccacgtgtgccgcctgctgggcatctgcctcacctccaccgtgcagctcatcatgcagctcatgccgtttggttgtctactcgactatgtccgggaacacaaagacaatattggctcccagtacctgctcaactggtgtgtgcagatcgcaaaggtaatcagggaag

SEQ ID NO: 6 - DNA, 188 bp, Artificial
  Synthetic ssODN EGFR-C797S
gatggccagcgtggacaacccccacgtgtgccgcctgctgggcatctgcctcacctccaccgtgcagctcatcatgcagctcatgcccttcgggagtctgcttgattacgtccgggaacacaaagacaatattggctcccagtacctgctcaactggtgtgtgcagatcgcaaaggtaatcagggaag

SEQ ID NO: 7 - DNA, 20 bp, Artificial
  Synthetic qPCR primer EGFR_Ctrl_FW
tgcttcccccattcaggact

SEQ ID NO: 8 - DNA, 20 bp, Artificial
  Synthetic qPCR primer EGFR_Ctrl_RV
ctccttgcacctcctcactg

SEQ ID NO: 9 - DNA, 22 bp, Artificial
  Synthetic qPCR primer EGFR_cbc_T790_RV
ccttccctgattacctttgcga

SEQ ID NO: 10 - DNA, 25 bp, Artificial
  Synthetic qPCR primer EGFR_cbc_T790T_FW
cacctctacagtccaactgattacc

SEQ ID NO: 11 - DNA, 28 bp, Artificial
  Synthetic qPCR primer EGFR_cbc_T790M_FW
ctctactgtacagcttataatgcaactg

SEQ ID NO: 12 - DNA, 21 bp, Artificial
  Synthetic qPCR primer EGFR_cbc_C797_RV
ccttatctcccctccccgtat

SEQ ID NO: 13 - DNA, 22 bp, Artificial
  Synthetic qPCR primer EGFR_cbc_C797C_FW
catgccgtttggttgtctactc

SEQ ID NO: 14 - DNA, 22 bp, Artificial
  Synthetic qPCR primer  EGFR_cbc_C797S_FW
cttcgggagtctgcttgattac